(19) United States
(12) Patent Application Publication
(10) Pub. No.: US 2005/0026230 A1
(43) Pub. Date: Feb. 3, 2005

(54) Method and apparatus for cell recovery

(76) Inventors: Yasuhiro Matsumura, Tokyo (JP); Hisayuki Matsushita, Nagareyama (JP); Hiroyuki Tsunoda, Kodaira (JP); Kunio Harada, Hachioji (JP)

Correspondence Address: ANTONELLI, TERRY, STOUT & KRAUS, LLP, 1300 NORTH SEVENTEENTH STREET, SUITE 1800, ARLINGTON, VA, 22209-9889, US

(21) Appl. No.: 10/773,316

(22) Filed: Feb. 9, 2004

(30) Foreign Application Priority Data
Jul. 29, 2003 (JP) 2003-281978

(51) Int. Cl. G01N 33/574; C12N 5/08

(52) U.S. Cl. 435/7.23; 435/366

(57) ABSTRACT

A method and apparatus for recovering cells from stool are provided for diagnosing colorectal cancer from stool naturally voided by multiple specimens. The method includes the steps of preparing a sample of naturally voided and collected stool, to which sample a buffer solution is added, causing cancer cells in the sample from which the impurities have been removed to be adsorbed on a solid carrier, and recovering the cancer cells thus adsorbed.

### BACKGROUND OF THE INVENTION

1. Field of the Invention

The present invention relates to a method of recovering cells from stool.

2. Background Art

In Europe and the United Sates, colorectal cancer is a leading cause of cancer deaths. In Japan too, the number of patients of colorectal cancer is increasing sharply in recent years. This is believed due to the more Westernized, meat-oriented diet adopted by the Japanese. About 60 thousand people in Japan are diagnosed with colorectal cancer every year. On an organ-by-organ basis, the number of deaths from colorectal cancer is third highest, following stomach and lung cancer, and the number is expected to increase. However, colorectal cancer is known to be almost 100% curable by operation if detected early. Thus, colorectal cancer is included in an early cancer screening scheme, and many examination methods have been developed.

Examples of the examination methods for the early detection of colorectal cancer include barium enema and colonoscopy. In an enema examination, barium is injected into the large intestine and allowed to attach to the mucous membrane surface of the intestine, so that the surface irregularities can be examined by X-ray. The colonoscopy involves the direct observation of the inside of the large intestine by an endoscope. These methods have high sensitivity and specificity to the detection of colorectal cancer. In addition, the colonoscopy has the advantage that early cancer or precancerous polyps can be removed. However, these examination methods put a large burden on the patients and are costly. Particularly, the colonoscopy requires skills in operation and is associated with the risk of complications such as bleeding or perforation. Accordingly, they are not suitable for screening the general public with no subjective symptoms for colorectal cancer.

As a method for the primary screening of the general public for colorectal cancer, a fecal occult blood test is widely used. In the fecal occult blood test, the presence or absence of bleeding in the bowel is detected by examining the presence of hemoglobin contained in the stool, in order to indirectly predict the development of colorectal cancer.

The fecal occult blood test can be roughly divided into two types, namely a chemical examination method and an immunological examination method. The chemical fecal occult blood test takes advantage of the peroxidase activity of hemoglobin, and it utilizes the reaction in which guaiac contained in a filter paper is turned into a blue-green oxide due to the active oxygen produced upon breakdown of hydrogen peroxide that is added as a matrix. Commercially available filter papers for such an examination include Hemoccult II filter paper (Fujisawa Pharmaceutical Co., Ltd.) and Shionogi B filter paper (Shionogi & Co., Ltd.).

The immunological fecal occult blood test utilizes the specific binding of antihuman hemoglobin antibody to human hemoglobin. The method is well on its way to becoming the dominant fecal occult blood test method due to its high specificity. Examples include a reversed-passive hemagglutination method (Immudia Hem-Sp, Fujirebio Inc.), a magnetic-particle agglutination and gradient method (Magstream Hem-Sp, Fujirebio Inc.), and a latex agglutination method (Immunoccult, Chugai Pharmaceutical Co., Ltd.).

Though the fecal occult blood test is widely used for the screening of colorectal cancer, some people are voicing suspicion over the efficacy of the test. A positive result in a chemical fecal occult blood test using the Hemoccult II requires 20 mg per day of bleeding in the large intestine. However, in an actual colorectal cancer patient, the amount of bleeding is thought to be 10 mg or less. As a result, the sensitivity of the fecal occult blood test is approximately 26%, and there have been reports that only about one quarter of the actual colorectal cancer patients can be detected and the remaining three quarters are overlooked (Jama, Vol. 269, 1262-7, 1993). Further, only 8.3% of all the positives actually had colorectal cancer and many false positives were included.

Thus, there is a need for the development of a new primary screening test method with better accuracy. As a possible candidate for that purpose, a testing method utilizing cancer cells shed onto the stool is gaining attention. Compared with the fecal occult blood test that detects the bleeding in the bowel which occurs indirectly in association with a colorectal cancer, the method of the present invention directly examines cancer cells and can therefore provide a more reliable testing method.

As a method of examining cancer cells in the stool, JP Patent Publication (Kohyo) No. 2002-515973 A (WO97/28450), for example, describes a method for genetic diagnosis utilizing nucleic acids directly extracted from stool. Concrete examples of the genetic mutation detecting method include the sequence method, the PCR-RFLP (polymerase chain reaction-restriction enzyme fragment length polymorphism) method, the SSCP (single-stranded conformational polymorphism) method, and the PTT (protein truncation test) method. Apart from the detection of genetic mutations, diagnostic methods that utilize the instability of a microsatellite (MSI, microsatellite instability) or the appearance of a long DNA (L-DNA) as indicators are known.

As the genes that can be examined for genetic mutations, K-ras, APC, P53 and DCC, for example, are widely known. Searches actively continue even now for genes that can be new objects of examination, taking advantage of different expression levels and utilizing a microarray, for example. A method has also been proposed that uses the expression pattern of a splicing variant of the CD44 gene as a marker.

One problem associated with these testing methods is the fact that the nucleic acids in the stool derive from various bacteria and normal cells, and that the ratio of genes deriving from cancer cells collected from the stool is very small (about 0.05%). This poses a significant hindrance in the examination of mutations in cancerous cell-derived genes or subtle changes in expression patterns, thus making the practical application of the method difficult.

Thus, methods of collecting cancer cells directly from stool and examining them have been considered, with a view to providing a more reliable colorectal cancer diagnosis. In order to collect cancer cells from stool, two steps are important. One is the step of exfoliating cells from the stool, and the other is the step of collecting the exfoliated cells.

JP Patent Publication (Kohyo) No. 11-511982 A (1999) (WO97/09600) reports a preliminary processing method whereby the stool is cooled in the step of exfoliating cells therefrom, and then the cells existing under the surface of the stool are exfoliated. Specifically, claim 1 of the document recites the step of “cooling the stool to a temperature below its gel freezing point,” and in this method, the surface of the stool cooled and frozen is scraped and the cancer cells existing in the surface are exfoliated. Methods have also been reported that employ a device called stomacher that is capable of mildly pulverizing a solid matter, wherein the entire stool is suspended and cells are exfoliated.

In the step of recovering the exfoliated cells, a centrifugal separation method utilizing a Percoll (Int J Cancer, Vol. 52, 347-50, 1992) or a recovery method utilizing magnetic beads to which an antihuman antibody is bound (Lancet, Vol. 359, 1917-9, 2002, Apmis, Vol. 110, 239-46, 2002) have been reported. Particularly, magnetic beads to which Ber-EP4 antibody is bound that specifically binds to epithelium cells are commercially available (Dynabeads Epithelial Enrich, Dynal Biotech), and they are known to bind to colorectal cancer cell lines. In Patent Document 2 too, the Ber-EP4 binding magnetic beads are utilized for recovering cancer cells from stool.

### SUMMARY OF THE INVENTION

In a large-scale colorectal cancer screening performed on the general public, a large amount of multiple specimens must be processed by an automated system. The method in which cells are directly recovered from stool and examined for colorectal cancer is excellent in reliability. However, in the existing method whereby cells are recovered from the surface of stool that is frozen by cooling, as described in JP Patent Publication (Kohyo) No. 11-511982 A (1999) (WO97/09600) (to be hereafter referred to as a cooling method), the operation is bothersome and the screening cannot be performed on a large scale.

In order to realize the automated processing of a large amount of multiple specimens, the operation must be simplified and the time of operation must be reduced. In the cooling method, there is the step of centrifugation, which takes time and does not render itself to automation and thus makes the processing of multiple specimens difficult. Further, the specimen cooling operation requires large-sized equipment and makes the stool processing operation complicated.

For the determination of colorectal cancer using the cells recovered from stool, a method employing a cytological analysis for identifying colorectal cancer is very effective. However, in the conventional cooling method, the cells are damaged by the cooling operation, thereby making it difficult to perform a cytological analysis.

Further, in the cooling method, the cells below the surface of the stool are exfoliated and recovered. In the ascending colon portion near the small intestine, the stool is in the form of muddy water, and it is believed that the most cancer cells exfoliated from the walls of the large intestine are later taken into the inner core of the stool during the process of forming a solid feces. Thus, it is likely that cancer cells deriving from the ascending colon cannot be recovered by the cooling method.

To solve the aforementioned problems, the method and apparatus for the recovery of cancer cells according to the invention are operated at room temperature.

Specifically, in contrast to the cooling method in which the surface of a cooled and frozen stool is scraped and then cancer cells existing in the surface of the stool are exfoliated, the method of recovering cells according to the invention includes the steps of, at room temperature, preparing a sample to which a buffer solution is added, causing a cancer cell in the sample from which the impurity has been removed to be adsorbed on a solid carrier, and recovering the thus adsorbed cancer cell. Thus, all of the steps for the recovery of cancer cells can be conducted without temperature control. Similarly, the apparatus for recovering cells according to the invention includes a bag for storing a sample comprising a buffer solution and stool at room temperature, and a container in which a solid carrier for the adsorption of a cell in the sample is stored. Thus, the cell recovery apparatus does not require a temperature control means. As a result, the cell recovering method and apparatus according to the invention can simplify the cell recovery operation and allow cancer cells in stool to be recovered stably and efficiently, thereby providing a high determination accuracy.

FIG. 1 shows the temperature dependency of the cell stability in stool and the antigen-antibody binding reaction rate. As shown in FIG. 1, when the temperature is low, the cell stability in stool decreases and, particularly, in a frozen state at temperatures of 4° C. or lower, the cancer cells could be destroyed, which would make it difficult to carry out the subsequent cytological analysis. On the other hand, with regard to the antigen-antibody binding reaction rate, the antibody is deactivated as the temperature increases, thus also making it difficult to carry out the subsequent immunological operations. Thus, in accordance with the invention, a range of temperatures around room temperature, or a temperature range between 5° C. and 40° C., or preferably between 15° C. and 35° C., is adopted so as to make both the cell stability and the antigen-antibody binding reaction rate compatible with each other. All of the steps from the recovery of cancer cells to the adsorption may be conducted at room temperature.

Further, the invention provides a novel filter system capable of handling a suspension of stool as a whole. A funnel-shaped filter improves the efficiency of filtering of a stool suspension, reduces the time required for operation, and eliminates the operation of centrifugation, thereby greatly simplifying the entire operation. Further, as cells are recovered from the stool as a whole including the central portion thereof, cancer diagnosis can be conducted on the entire large intestine. On the other hand, a multi-stage filtering apparatus allows the cells to be trapped on a film and thus condensed. The film in which the cells are trapped can then be recovered, so that the invention can be adapted for an automated system.

Further, conditions for a protocol have been analyzed, including the addition of blood serum into a stool suspension, and a simplified protocol has been developed whereby all of the steps can be operated at room temperature.

In accordance with the method and apparatus of the invention for cell recovery, good, living cancer cells can be recovered from stool at room temperature, in contrast to the cooling method as disclosed in JP Patent Publication (Kohyo) No. 11-511982 A (1999) (WO97/09600), whereby the surface of a cooled and frozen stool is scraped and then cancer cells existing in the surface of the stool are exfoliated. Thus, in accordance with the invention, the recovered cells can be subjected to cytological, immunological and biochemical analyses with high accuracy. Also, the method and apparatus of the invention for recovering cells can utilize a cell deriving from early colorectal cancer or the stool as a whole as a specimen, cancer cells deriving from the ascending colon, which are difficult to detect endoscopically, can be recovered. Thus, the invention can provide a highly reliable examination method. Further, the method and apparatus of the invention have eliminated the centrifugation and cooling operations so that the operation can be simplified and performed in less time. Thus, an automated total system for colorectal cancer examination can be constructed using the method and apparatus of the invention.

### BRIEF DESCRIPTION OF THE DRAWINGS

FIG. 1 shows a graph indicating the temperature dependency of the cell stability in stool and the antigen-antibody binding reaction rate.

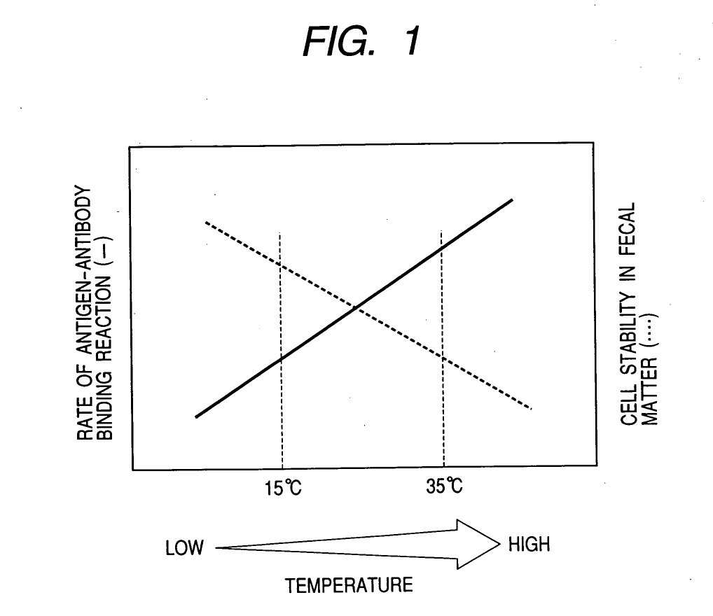

FIG. 2 schematically shows a standard protocol for a method of recovering colorectal cancer cell from stool.

FIG. 3 shows the shape of a funnel-shaped filter for filtering a stool suspension.

FIG. 4 shows the concept of a total system for colorectal cancer examination according to the present invention.

FIG. 5 shows the results of analysis of conditions for the cell recovery method. FIG. 5(A) shows the relationship between the temperature at which the cells and the magnetic beads bind to one another, and the cell recovery rate. FIG. 5(B) shows the relationship between the presence or absence of blood serum in the medium and the cell recovery rate.

FIG. 6 compares the cell recovery rate according to the conventional method and that according to the magnetic bead method of the invention.

### DESCRIPTION OF THE PREFERRED EMBODIMENTS

(Recovery of Cells from Stool)

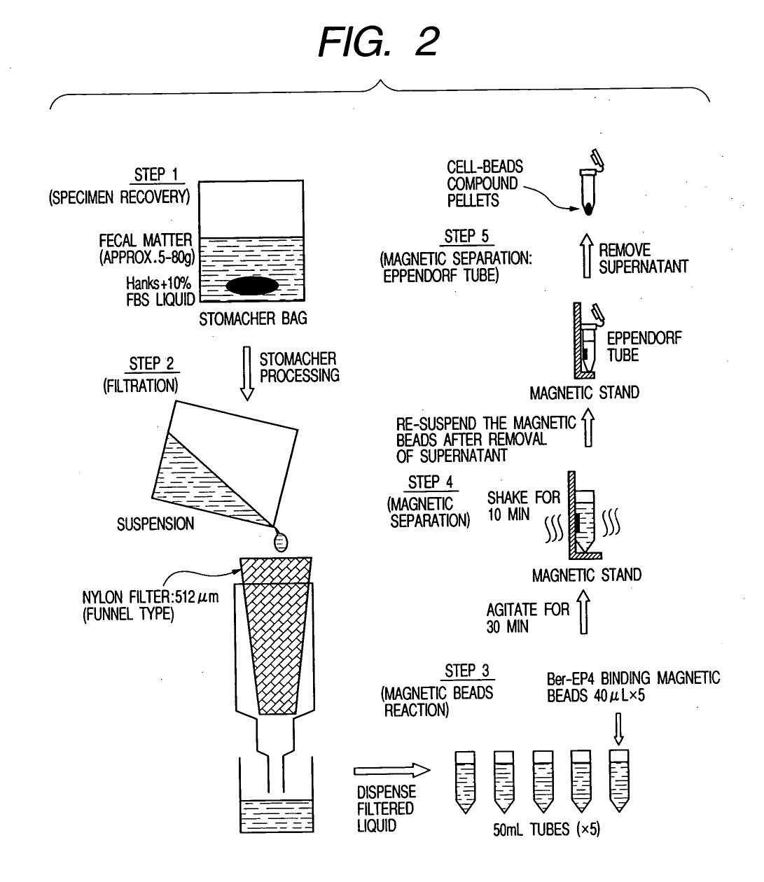

FIG. 2 shows the standard protocol for the method of recovering cells from stool according to the present invention. Hereafter, the procedure of the standard protocol will be described.

(Step 1: Recovery of Specimens)

The stool used in the present invention is stool naturally voided by a human. A solid stool is used and diarrheal stool is not used. Neither is that stool used that has been voided after the subject had taken a compulsive relieving agent such as a laxative or barium for intestinal examination. There is no need for the subject to exercise any particular diet control prior to the test.

The stool for specimen is recovered on a dish- or sheet-shaped disposable container, and an appropriate amount is put into a stomacher bag. Other methods of stool recovery may be employed, such as by means of a stick-type stool recovery apparatus or a stamp-type recovery apparatus, as long as the method is capable of recovering an appropriate amount of stool. As the stomacher bag, a commercially available filterless bag is employed. A stomacher bag with a filter may be used. The stomacher herein refers to mixers in general for the pulverization of specimens contained in a bag-like container. The stomacher bag refers to a commercially available bag specifically designed for stomachers. Other substitutes may be used as long as they can be used with stomachers.

The stool recovered from the subject should preferably be used within three hours but may be used within up to approximately ten hours. The stool can be stored at room temperature during that period and there is no need to store it in a refrigerated or frozen state.

The amount of stool used is preferably in the range between about 5 g and 80 g. However, the amount may range from about 0.5 g to 200 g.

A suspension medium is put into the stomacher bag in which the stool is recovered. The medium is a Hanks solution. However, any conventional medium that is used in experiments involving cells may be used. Specific examples include PBS, PBS (−) and media for the cultivation of various cells (MEM, DMEM, RPMI).

The amount of medium added may be varied depending on the amount or state of stool. However, the amount is preferably 1 mL or more per 1 g of stool. By adding 200 mL of medium per one stomacher bag, any of the aforementioned amounts of stool may be accommodated.

Blood serum is added to the medium. The concentration of blood serum is preferably 10% but may be in the range between 0.5% and 20%. The blood serum is preferably FBS (fetal bovine serum) but may be CS (calf serum).

The stomacher bag containing the stool with medium is sealed with a sealer. Leakage of the suspension can be more completely prevented by covering the stomacher bag with another stomacher bag. The thus sealed stomacher bag is processed by a stomacher to produce a stool suspension. This step of recovering a specimen is conducted at room temperature. However, if it takes time between the recovery of specimen and filtration, the stool suspension may be stored in a cooler box, for example.

(Step 2: Filtration)

The suspension is filtered by a filter in a draft to remove residual matter. When a stomacher bag with a filter is used, the suspension is filtered by the filter in the bag and the filtered solution is recovered.

When a stomacher bag with no filter is used, a new filtering apparatus is used for filtering the suspension.

The filtering apparatus is used either with a single filter or in a multiple-stage arrangement made up of filters with various sizes. When a single filter is used, the size is preferably about 500 μm. However, the size may be in the range between 40 to 1500 μm, or preferably between 400 and 1000 μm. When the multiple-stage arrangement is used, the suspension is caused to flow from a filter with a larger size to one with a smaller size. The size of the filters for the multiple-stage filtering may be between about 40 to 2000 μm. By disposing a filter with a size of 10 μm or less in the final stage, the cells can be captured on the final filter.

The filtering apparatus may be either of a free-fall type or a suction filtering type.

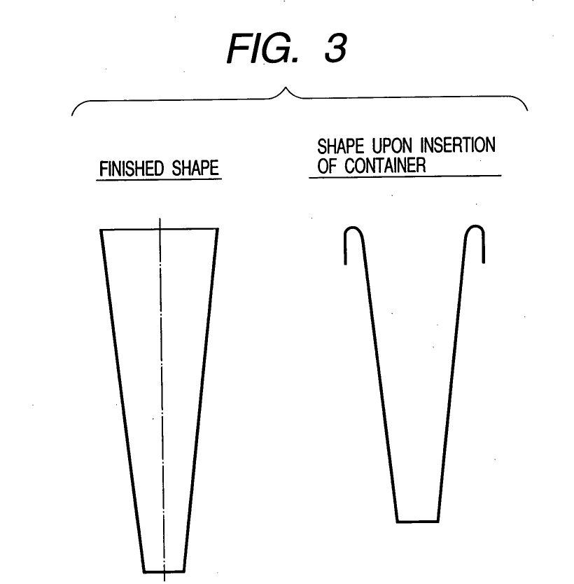

FIG. 3 shows an example of the shape of filter. The funnel shape of the filter has dimensions such that the diameter of the opening is 60 mm, the diameter of the bottom is 20 mm, the height is 200 mm, and the height when the filter is inserted into a container is 170 mm, for example. The filter shown in FIG. 3 is a funnel-type three-dimensional filter having filtering sides. Preferably, a bottom-surface type filter having a filtering surface only at the bottom may be used. Further, the surface of the filter may be provided with fold-like irregularities such that the area of contact with the suspension can be increased.

The material of the filter is preferably nylon. Other materials may be used as long as they are capable of allowing a filter with a desired size or shape to be produced. Specific examples include polyester, polyethylene, and polypropylene. This filtering step is conducted at room temperature.

(Step 3: Magnetic Beads Reaction)

The cells contained in the filtered solution are recovered using a carrier having an affinity for cancer cells. The carrier is made of magnetic beads having bound to the surfaces thereof an antibody with an affinity for cancer cells. Specifically, Ber-EP4 antibody-binding magnetic beads commercially available from Dynal Biotech (Dynabeads Epithelial Enrich) are used. Besides Ber-EP4, other antibodies having an affinity for colorectal cancer cells may be used. Besides antibodies, aptamars or ligands having an affinity for colorectal cancer cells may be used.

Forty mircrolitters of magnetic beads are added per tube containing about 20 to 45 mL of dispensed filtered solution. The amount of magnetic beads may be varied between about 20 μL and 400 μL.

The filtered solution to which the magnetic beads have been added is blended using a mix rotor, such that the cells in the filtered solution are bound to the magnetic beads. The blending is preferably conducted at room temperature or in a cold room at 4° C., preferably for 10 minutes or more. This step of reacting magnetic beads is carried out at room temperature.

(Step 4: Magnetic Separation)

The tubes containing the blended filtered solution are placed on a magnetic stand and is then shook for 15 minutes, such that the magnetic beads are collected on the side of the tube. The shaking is preferably conducted for 10 minutes or more. The shaking may be conducted in any manner, such as a see-saw motion, rotation or gyration, as long as the filtered solution can be gradually blended.

After the magnetic beads have attached to the wall surface, the filtered solution is removed. After the removal of solution, the tubes are detached from the magnetic stand and washed with the aforementioned medium, and then the beads washing solution is recovered. The amount of medium is 500 μL per tube but may be varied as desired in light of the subsequent experiment. This step of magnetic separation is conducted at room temperature.

(Step 5: Magnetic Separation, Eppendorf Tube)

The washing solution is recovered into an Eppendorf tube or the like smaller than the previously used tubes. The tube containing the washing solution is immediately attached to a dedicated magnetic stand. After the magnetic beads have been collected on the side walls of the Eppendorf tube, the supernatant is removed to obtain pellets of cell-bead complexes. This step of magnetic separation and Eppendorf tube is conducted at room temperature.

(Diagnosis for Colorectal Cancer)

The pellets recovered by the present standard protocol are then used as specimens for the determination of colorectal cancer. The determination of cancer is based either on cells themselves or a substance extracted from cells. When cells themselves are used, the pellets are used immediately after recovery. When an extracted substance is used, the pellets can be stored in a frozen state at −80° C.

When cells themselves are used, the cells are stained with Papanicolaou stain and then observed by a microscope for determination. If the ratio of nucleus to cytoplasm (N/C) is high and atypical cells with chromatin condensation are identified, the cells are determined to be cancerous. Other staining methods may be used as long as they are capable of identifying cancer cells. Besides conventional staining, immunostaining that utilizes a cancer-cell specific antibody may be used.

It is possible to extract DNA or RNA from cells and utilize them for cancer determination. For the extraction of DNA or RNA, nuclear acid extraction kits available from various companies can be employed. Specific examples of such kits include Dynabeads DNA DIREIC Universal from Dynal Biotech, QIAamp DNA MiniKit from Qiagen, and SepaGene from Sanko Junyaku Co., Ltd. for DNA extraction. For the extraction of RNA, ISOGEN from Nippon Gene Co., Ltd. and TRIzol Reagent from Invitrogen can be cited. The extracted nucleic acids can be utilized for the various methods mentioned in the description of the related art.

(Stool Processing Total System)

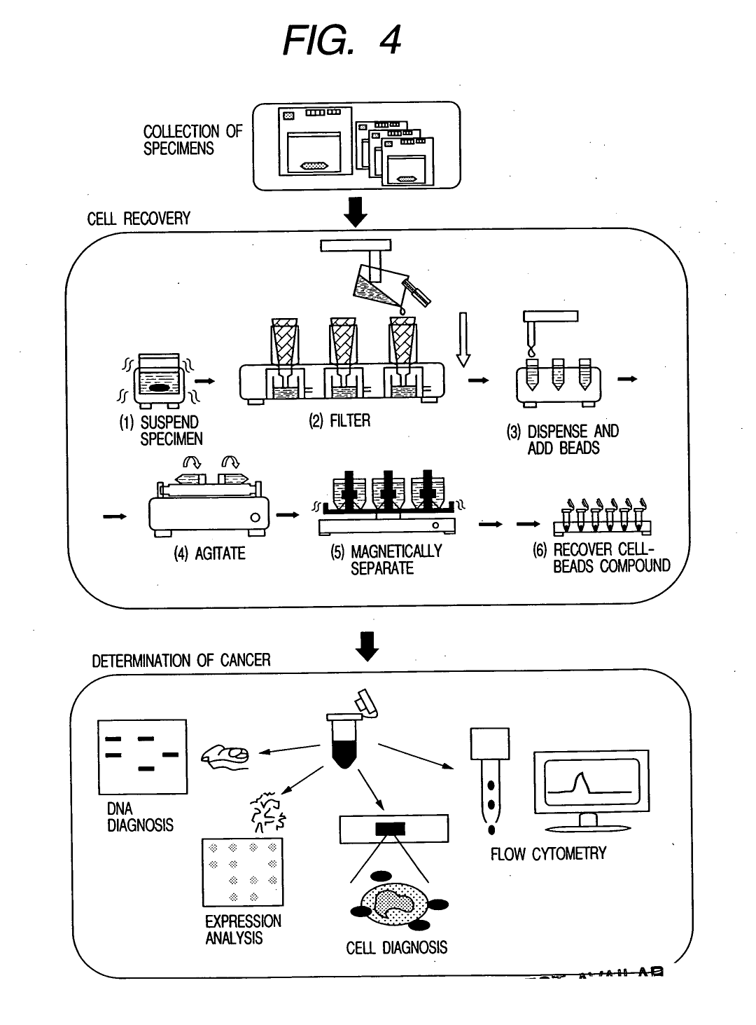

The concept of a stool processing total system according to the present invention is shown in FIG. 4. Collected specimens are suspended using a stomacher. For the filtering of the suspension, an apparatus in which single funnel-shaped filters are arranged as shown in FIG. 4 can be used. The apparatus may be replaced with a multi-stage filtering apparatus. The filtering apparatus is equipped with a suction filtering function. The filtered liquid is dispensed and agitated after the addition of beads. For the agitation, a conventional agitator may be adapted, or an apparatus suitable for the simultaneous processing of multiple specimens may be used. For magnetic separation, a conventional magnetic stand or a stand with an increased magnetic force adapted for the processing of multiple specimens may be used.

Cancer determination is made by using the recovered cell-beads complex. For the determination of cancer, a material extracted from the cells or the cells themselves are used. An automated system is constructed by adapting examination methods based on expression analysis utilizing a DNA chip or protein chip, or the identification of cancer cells utilizing flow cytometry.

### EXAMPLES

Hereafter, an example of the invention will be described. The invention, however, is not limited to the example.

(Recovery of Cells from Stool)

Stool voided by a colorectal cancer patient prior to operation was used as a specimen. As to the use of stool, the subject was informed of the content of the procedure prior to the experiment and a written consent was obtained.

Two hundred mL of Hanks solution (Nissui) containing 10% FBS was put into a stomacher bag containing stool (about 5 to 80 g) and the bag was then sealed. A stool suspension was then prepared by using a stomacher (200 rpm, 1 min).

In the case where a stomacher bag having a filter was used, the suspension was filtered by the filter in the bag. In the case where a stomacher bag without a filter was used, the suspension was filtered by passing it through a funnel-shaped filter set on a tubular plastic container. The filtered solution was recovered in a beaker. The filtered solution was further dispensed into five 50-mL centrifugal sedimentation tubes.

Forty μL of Ber-EP4 antibody-binding magnetic beads (Dynabeads Epithelial Enrich, Dynal Biotech) was added per centrifugal sedimentation tube, and the mixture was blended using a mix rotor (VMR-5, available from AS ONE) (at 4° C., 60 rpm, 30 min) so that the cells in the filtered solution were bound to the Ber-EP4 antibody.

After the individual centrifugal sedimentation tubes were set on a magnetic stand (Dynal MPC-1, Dynal Biotech), the stand was laid on a mild mixer (SI-36, TAITEC Corporation) horizontally. The tubes were then subjected to a seesaw motion for 15 minutes (60 reciprocations per min) to blend the filtered solution such that the magnetic beads were collected on the side walls of the centrifugal sedimentation tubes.

After the filtered solution was removed, the centrifugal sedimentation tubes were detached from the stand, and 500 μL of Hanks solution containing 10% FBS was added per tube to wash the beads collected on the wall surface.

The washing solution containing the beads was recovered into five Eppendorf tubes (1.5 mL each) in which 500 μL of Hanks solution containing 10% FBS had been put in advance. After a light suspension, the tubes were set on a magnetic stand (Dynal MPC-S, Dynal Biotech), and the magnetic beads were collected on the side walls of the Eppendorf tubes.

After the removal of the washing solution, the Eppendorf tubes were detached from the stand, and 1 mL of Hanks solution containing 10% FBS was added to each tube, and then the beads collected on the wall surface were washed. Similarly, the tubes were set on the magnetic stand and the magnetic beads were collected on the side walls of the Eppendorf tubes. The supernatant was then removed to obtain pellets of cell-beads complex. The recovery was conducted at room temperature.

(Cytological Analysis of the Recovered Cells)

The cell-beads complex pellets in each tube recovered in Example 1 were suspended with the addition of 100 μL of YM fixing solution. The suspension was then transferred to a 50-mL centrifugal sedimentation tube. The total amount was adjusted to 25 mL by YM fixing solution, thereby obtaining a cell-containing fixing solution. The cell-containing fixing solution was then dispensed into an automatic smearing apparatus for 8 glass slides. The apparatus was further filled with the YM fixing solution and was then centrifuged at 2000 rpm for 10 minutes, thereby smearing the glass slides with the cells. After drying the slides with cold air, the cells were fixed with 95% ethanol.

The cells were then stained by the Papanicolaou staining method, which is a representative method of staining for the observation of cell morphology. The presence or absence of cancer cells were microscopically observed and determined. The results are shown in Table 1.

| | | Dukes | Cyto- | |
|---|---|---|---|---|
| No. | Tumor site¹⁾ | classification | diagnosis | Remarks²⁾ |
| 1 | S | C | + | |
| 2 | Ra | B | + | |
| 3 | S | A | − | Diarrhea due |
| | | | | to Niflec |
| 4 | Rb | C | − | Diarrhea due |
| | | | | to Niflec |
| 5 | Rb | D | − | Clogged filter |
| 6 | S | A | − | Clogged filter |
| 7 | Ra | C | + | |
| 8 | Rb | A | + | |
| 9 | Rb | A | + | |
| 10 | Ra | A | + | |
| 11 | Rs | A | + | |
| 12 | Ra | A | + | |
| 13 | Rb | A | + | |
| 14 | A | A | + | |
| 15 | S | C | + | |
| 16 | T | A | + | |

¹⁾5: Sigmoid colon
Ra: Upper rectum
Rb: Lower rectum
Rs: S-shaped portion of rectum
A: Ascending colon
T: Transverse colon
2)Nos. 1-12: Stomacher with a filter was used. Nos. 13-16: Funnel-shaped filter was used.

Table 1 shows the cases of colorectal cancer patients who provided the specimens used in the present experiment. Cytodiagnosis (+) indicates the cases where cancer cells were collected by the method of the invention, and cytodiagnosis (−) indicates the cases where no recovery of cancer cells were confirmed by the inventive method.

In cases Nos. 3 and 4, Niflec, a laxative, had been taken by the patient prior to the passage of stool, so that the voided stool was diarrheal from which cells could not be recovered. In cases Nos. 5 and 6 too, no cells could be recovered. In these two cases, the amount of stool exceeded 100 g and, in addition, a stomacher bag with a filter was used at this point for filtering the stool suspension, resulting in a significant clogging of the filter. The reduction in the cell collection rate depending on the extent of clogging of the filter had also been anticipated in a preliminary experiment, and it was clearly shown that an excessive clogging of the filter would prevent the recovery of cells.

Thus, cells were recovered from 12 out of 16 cases (75%). Even in cases where no cell could be recovered, the reasons for the absence of recovery were obvious, as mentioned above. Thus, it was clearly shown that cells can be very efficiently recovered from colorectal cancer patients by using the magnetic beads method in accordance with the present protocol.

When the progress of cancer in the colorectal cancer patients who provided the specimens was examined, 8 out of the 12 cases from which cells were recovered (67%) were classified into early cancer of Dukes A. In addition, cells could be recovered from cases of Dukes A involving the tumor sites of upper rectum (No. 14) or transverse colon (No. 16). These results show that the method of the invention is very effective in diagnosing early cancer including those cancers developed at sites where detection by colonoscopy, for example, is difficult.

(Analysis of the Conditions for the Magnetic-Beads Cell Recovery Method Using Cultured Cells)

A colorectal cancer cultured cell line (HT-29) was mixed in a suspension of stool collected from an infant, and the mixture was reacted with a Ber-EP4 antibody-binding magnetic bead. The conditions that affect the cell recovery rate in the present method were then examined. The suspension of the stool from infant was filtered by the above-described funnel-shaped filter to obtain a filtered solution. The recovery rate was calculated by measuring the number of cells that bound to the recovered beads using Nucleo Counter (from M&S TechnoSystems) and comparing it with the number of cells initially added to the suspension.

First, the temperature suitable for the binding of beads and cells was examined. Magnetic beads and cells are in many cases reacted at 4° C. This is for the purpose of lowering the damage done to the cells or preventing the phenomena in which macrophages contained in the specimen prey on the beads, for example.

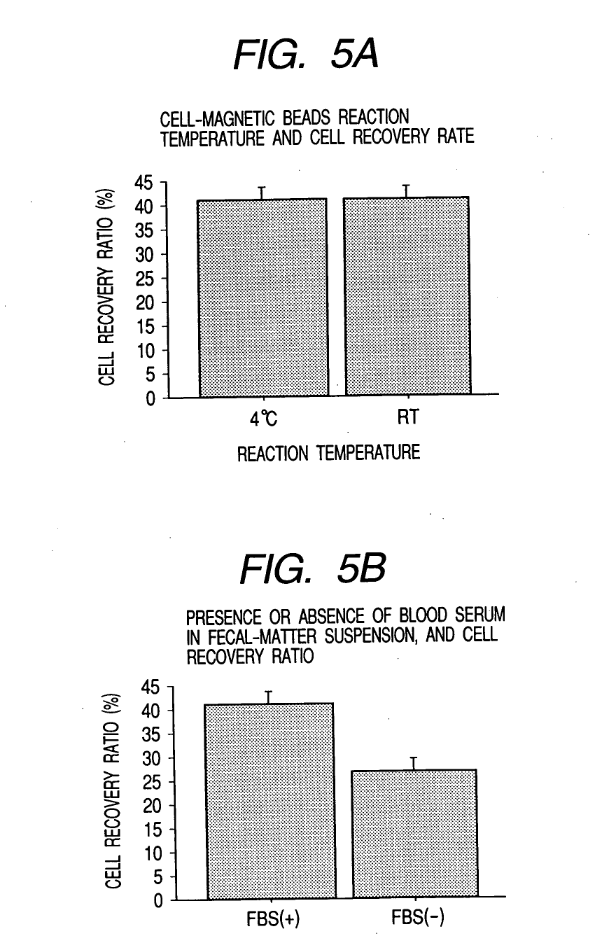

Thus, a mixing reaction between a 25-mL cell-(8.4×10⁵) stool suspension and 40 μL of beads was conducted at 4° C. and at room temperature. As a result, it was revealed that the same level of cell recovery rate can be obtained at room temperature as at 4° C. (FIG. 5A). This indicates that all of the cell recovery steps of the method of the invention can be conducted at room temperature, whereby individual operations can be greatly simplified as compared with the aforementioned cooling method.

Next, the need for the blood serum in the cultured solution for suspension was analyzed. Blood serum was expected to have functions for improving the efficiency of the magnetic beads method by, for example, restricting the protease activity in the solution and stabilizing the cells, or preventing the adsorption of non-specific cells.

Accordingly, 25 mL of cell-stool suspension was prepared using a Hanks solution containing 10% blood serum (FBS, fetal bovine serum) and a Hanks solution containing no blood serum, and the suspension was reacted with 40 μL of beads, in order to examine the cell recovery rate in a similar manner.

The results (FIG. 5B) showed that the cell recovery rate was lower in the case where no blood serum was contained, thus indicating the effectiveness of blood serum in the recovery method of the invention.

Further, the size of the mesh of the funnel-shaped filter used in the present experiment was analyzed. Nylon filters of different sizes (1000, 512, 96 and 48 μm), with the entire surface made of mesh and a shape as shown in FIG. 3, were prepared. Cultured cells were added to a stool suspension in a similar manner, filtered by these four kinds of filters, and then reacted with beads to examine the cell recovery rate.

As a result, the recovery rate of the filter of 96 μm was about one half of that of 512 μm, and the rate for 48 μm was about one fifth of that for 512 μm. The filter of 1000 μm had approximately the same recovery rate as that of the filter of 512 μm. These results thus indicated that the size of filter should preferably be 500 or more. However, in order for the filter to function as such, the size should not exceed 1500 μm.

(Comparison with the Percoll Centrifugation Method)

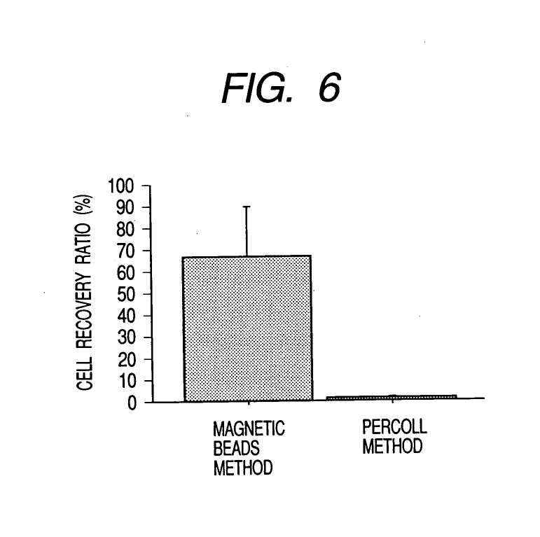

The cell recovery rate of the magnetic beads method was compared with that of the Percoll centrifugation method, which is a conventional method. The experiment was conducted using cultured cells in a manner similar to those described above. The Percoll centrifugation method was carried out in accordance with the method of Yamao et al. reported in Non-Patent Document 4. In the Percoll centrifugation method, Percoll solution is mixed with cells and is then centrifuged in order to separate the cells according to their densities. The results are shown in FIG. 6. The cell recovery rate was 0.8% for the Percoll centrifugation method. The recovery rate for the magnetic beads method (in accordance with the standard cell recovery protocol was 66.7%, thus indicating the advantage of the magnetic beads method.

1. A method of recovering a cancerous cell comprising the steps of:
preparing a sample comprising a collected naturally voided stool to which a buffer solution is added, at room temperature;
causing a cancer cell in said sample from which said impurity has been removed to be adsorbed on a solid carrier; and
recovering the thus adsorbed cancer cell.

2. The method according to claim 1, further comprising the step of removing an impurity from said sample using a filter.

3. The method according to claim 1, wherein all of the steps for the recovery of a cancerous cell are conducted without temperature control.

4. The method according to claim 1, wherein the step of causing the cancerous cell in the sample from which impurity has been removed to be adsorbed on the solid carrier and the step of recovering the adsorbed cancerous cell are also conducted at room temperature.

5. The method according to claim 1, wherein said buffer solution contains blood serum.

6. The method according to claim 1, wherein the amount of said buffer solution is equal to or more than the amount of said stool.

7. The method according to claim 1, wherein on the surface of said solid carrier is immobilized an antibody against an antigen on the surface of an epithelial cell and/or epithelial cancer cell.

8. The method according to claim 1, wherein said solid carrier is a magnetic bead, and wherein the recovery is carried out using a magnet.

9. The method according to claim 1, wherein said room temperature is not less than 15° C. and not more than 35° C.

10. A cell recovery apparatus comprising:
a bag for storing a sample comprising a buffer solution and stool at room temperature; and
a container in which a solid carrier for the adsorption of a cell in said sample is stored.

11. The cell recovery apparatus according to claim 10, further comprising:
a filter portion connected to said bag for removing an impurity in said sample, wherein said container storing said solid carrier for the adsorption of cell in said sample is connected to said filter portion.

12. The cell recovery apparatus according to claim 11, wherein said filter portion is made up of one or more filters with different coarseness.

13. The cell recovery apparatus according to claim 10, wherein said container is provided with an agitating means for agitating at least one of said solid carrier and said sample.

14. The cell recovery apparatus according to claim 10, wherein said solid carrier is a magnetic bead, and wherein a recovering magnet is further provided near said container.

15. The cell recovery apparatus according to claim 10, wherein no temperature control means is provided.
